(19) United States
(12) Patent Application Publication
(10) Pub. No.: US 2008/0077016 A1
(43) Pub. Date: Mar. 27, 2008

(54) MONITORING SYSTEM HAVING IMPLANTABLE INDUCTIVE SENSOR

(75) Inventors: Douglas Ray Sparks, Whitmore Lake, MI (US); Nader Najafi, Ann Arbor, MI (US)

Correspondence Address: HARTMAN & HARTMAN, P.C., 552 EAST 700 NORTH, VALPARAISO, IN, 46383, US

(73) Assignee: INTEGRATED SENSING SYSTEMS, INC., Ypsilanti, MI (US)

(21) Appl. No.: 11/860,010

(22) Filed: Sep. 24, 2007

Related U.S. Application Data
(60) Provisional application No. 60/846,280, filed on Sep. 22, 2006.

(51) Int. Cl. A61B 5/103

(52) U.S. Cl. 600/459

(57) ABSTRACT

A system for monitoring physical properties of structures within animate and inanimate objects, including internal organs and bones of humans. The system includes sensing and readout devices. The sensing device is adapted to be implanted in a body and attached to a structure within the body, and includes an electrical circuit containing a first inductor coil formed at least in part by a conductor with portions thereof separated by gaps. The first inductor coil is adapted to be physically coupled to the structure so that changes in shape and size of the structure cause changes in shape and/or size of the first inductor coil and/or changes in the gaps, which alter the inductance of the first inductor coil when current flows through the electrical circuit. The readout device is not adapted to be implanted in the patient, and includes an inductor coil capable of electromagnetic telecommunication and/or electromagnetic powering of the sensing device.

### CROSS REFERENCE TO RELATED APPLICATIONS

This application claims the benefit of U.S. Provisional Application No. 60/846,280, filed Sep. 22, 2006, the contents of which are incorporated herein by reference.

### BACKGROUND OF THE INVENTION

The present invention generally relates to sensing devices and systems. More particularly, this invention relates to a system for monitoring physical properties of structures within animate and inanimate objects, for example, changes in shape, size, etc., of an internal organ, bone, etc., of a human.

Wireless devices such as pressure sensors have been implanted and used to monitor heart, brain, bladder and ocular function. For example, see commonly-assigned U.S. Pat. Nos. 6,926,670 and 6,968,734 to Rich et al., and N. Najafi and A. Ludomirsky, “Initial Animal Studies of a Wireless, Batteryless, MEMS Implant for Cardiovascular Applications,” Biomedical Microdevices, 6:1, p. 61-65 (2004). With such technologies, pressure changes are sensed with an implant equipped with a mechanical capacitor (tuning capacitor) having a fixed electrode and a moving electrode, for example, on a diaphragm that deflects in response to pressure changes. The implant is further equipped with an inductor in the form of a fixed coil that serves as an antenna for the implant, such that the implant is able to receive radio frequency (RF) signals from the outside world and transmit the frequency output of the circuit.

FIGS. 1a and 1b represent two types of wireless pressure sensing approaches disclosed in the Rich et al. patents. In FIG. 1a, an implant 10 is shown as operating in combination with a non-implanted external reader unit 20, between which a wireless telemetry link is established using a resonant scheme. The implant 10 contains a packaged inductor coil 12 and a pressure sensor in the form of a mechanical capacitor 14. Together, the inductor coil 12 and capacitor 14 form an LC (inductor-capacitor) tank resonator circuit that has a specific resonant frequency, expressed as 1/(LC)^{1/2}, which can be detected from the impedance of the circuit. At the resonant frequency, the circuit presents a measurable change in magnetically-coupled impedance load to an external coil 22 associated with the reader unit 20. Because the resonant frequency is a function of the capacitance of the capacitor 14, the resonant frequency of the LC circuit changes in response to pressure changes that alter the capacitance of the capacitor 14. Based on the coil 12 being fixed and therefore having a fixed inductance value, the reader unit 20 is able to determine the pressure sensed by the implant 10 by monitoring the resonant frequency of the circuit.

FIG. 1b also shows a wireless pressure sensor implant 30 operating in combination with a non-implanted external reader unit 50. A wireless telemetry link is established between the implant 30 and reader unit 50 using a passive, magnetically-coupled scheme, in which on-board circuitry of the implant 30 receives power from the reader unit 50. In the absence of the reader unit 50, the implant 30 lays passive and without any internal means to power itself. When a pressure reading is desired, the reader unit 50 must be brought within range of the implant 30.

In the embodiment of FIG. 1b, the implant 30 contains a packaged inductor coil 32 and a pressure sensor in the form of a mechanical capacitor 34. The reader unit 50 has a coil 52 by which an alternating electromagnetic field is transmitted to the coil 32 of the implant 30 to induce a voltage in the implant 30. When sufficient voltage has been induced in the implant 30, a rectification circuit 38 converts the alternating voltage on the coil 32 into a direct voltage that can be used by electronics 40 as a power supply for signal conversion and communication. At this point the implant 30 can be considered alert and ready for commands from the reader unit 50. The implant 30 may employ the coil 32 as an antenna for both reception and transmission, or it may utilize the coil 32 solely for receiving power from the reader unit 50 and employ a second coil 42 for transmitting signals to the reader unit 50. Signal transmission circuitry 44 receives an encoded signal generated by signal conditioning circuitry 46 based on the output of the capacitor 34, and then generates an alternating electromagnetic field that is propagated to the reader unit 50 with the coil 42.

In addition to monitoring heart, brain, bladder, and ocular function, capacitive sensors as discussed above have been proposed for monitoring joint pressure and orthopedic conditions, and have been further proposed for monitoring bone integrity when coupled to a resistive strain gauge, accelerometer or optical fibers. For example, see U.S. Pat. Nos. 5,425,775, 5,792,076, 6,034,296, 6,712,778, and 7,097,662. Notwithstanding such advancements, there is an ongoing desire for implantable sensors that can provide additional sensing capabilities.

### BRIEF SUMMARY OF THE INVENTION

The present invention provides a system for monitoring physical properties of structures within animate and inanimate objects, including but not limited to changes in shape, size, etc., of an internal organ or bone of a person.

The monitoring system includes at least one sensing device and a readout device. The sensing device is adapted to be implanted in a body and attached to a structure within the body. The sensing device includes an electrical circuit containing at least a first inductor coil formed at least in part by a conductor with portions thereof separated by gaps. The first inductor coil is adapted to be physically coupled to the structure so that changes in shape and size of the structure cause changes in shape and/or size of the first inductor coil and/or changes in the gaps so as to alter the inductance of the first inductor coil when current flows through the electrical circuit. The readout device is not adapted to be implanted in the patient, and includes at least one inductor coil and telemetric means for electromagnetic telecommunication and/or electromagnetic powering of the sensing device with the inductor coil.

In view of the above, it can be seen that the invention provides a telemetric monitoring system for noninvasively monitoring parameters associated with conditions surrounding the implantable sensing device, including conditions that reflect the health or a condition of a person or structure in which the sensor is implanted. In addition, the use by this invention of a variable inductor as a sensing element offers significant advantages, including a larger sampling area and volume that provides the ability to monitor larger objects than possible with variable capacitive sensors, which are generally limited to sensing pressure in an immediately surrounding fluid. Variable inductive sensing elements used with this invention are also more readily capable of sensing certain conditions in comparison to variable capacitive sensors, including the ability to sense strain, stress, swelling, rupture, cracking, etc., of a wide variety of structures and bodies, including but not limited to internal organs and bones and joints (both natural and artificial). Furthermore, a variable inductive sensing element can be applied to a limited region of a structure to sense localized conditions, or envelop the entire structure. Variable inductive sensing elements that encircle a bladder, organ, bone, joint, etc., will also typically have a larger diameter than that possible for a fixed on-chip inductor coil used in the prior art, and as such will have a longer transmission range than the coils of the prior art.

Other objects and advantages of this invention will be better appreciated from the following detailed description.

### BRIEF DESCRIPTION OF THE DRAWINGS

FIGS. 1a and 1b are schematic representations of prior art wireless pressure monitoring systems adapted for, respectively, resonant and passive sensing schemes.

FIGS. 2a and 2b are schematic representations of wireless monitoring systems adapted for, respectively, resonant and passive sensing schemes using an inductive sensing coil in accordance with embodiments of this invention.

FIGS. 3a and 3b schematically represent a plan view and a cross-sectional view, respectively, of a sensing coil configuration suitable for use with the monitoring systems of FIGS. 2a and 2b.

FIGS. 4 and 5 schematically represent plan views of two alternative sensing coil configurations suitable for use with the monitoring systems of FIGS. 2a and 2b.

FIG. 6 schematically represents the sensing coil of FIG. 5 wrapped around a pliable internal body structure for sensing changes in the shape, size, etc., of the structure.

FIGS. 7 and 8 schematically represent the sensing coil of FIG. 5 embedded in surfaces of an artificial ball joint and artificial patella, respectively.

FIG. 9 schematically represents the use of two sensing coils of the type represented in FIG. 5 embedded in surfaces of an artificial ball joint.

FIG. 10 schematically represents the sensing coil of FIG. 5 embedded in a surface of an artificial ball joint that contains a metal core.

FIGS. 11 and 12 schematically represent two circuits containing sensing coils, and further including, respectively, a variable resistor and an integrated circuit.

FIG. 13 schematically represents the circuit of FIG. 11 applied as a flexible thin-film sensor to a bone.

### DETAILED DESCRIPTION OF THE INVENTION

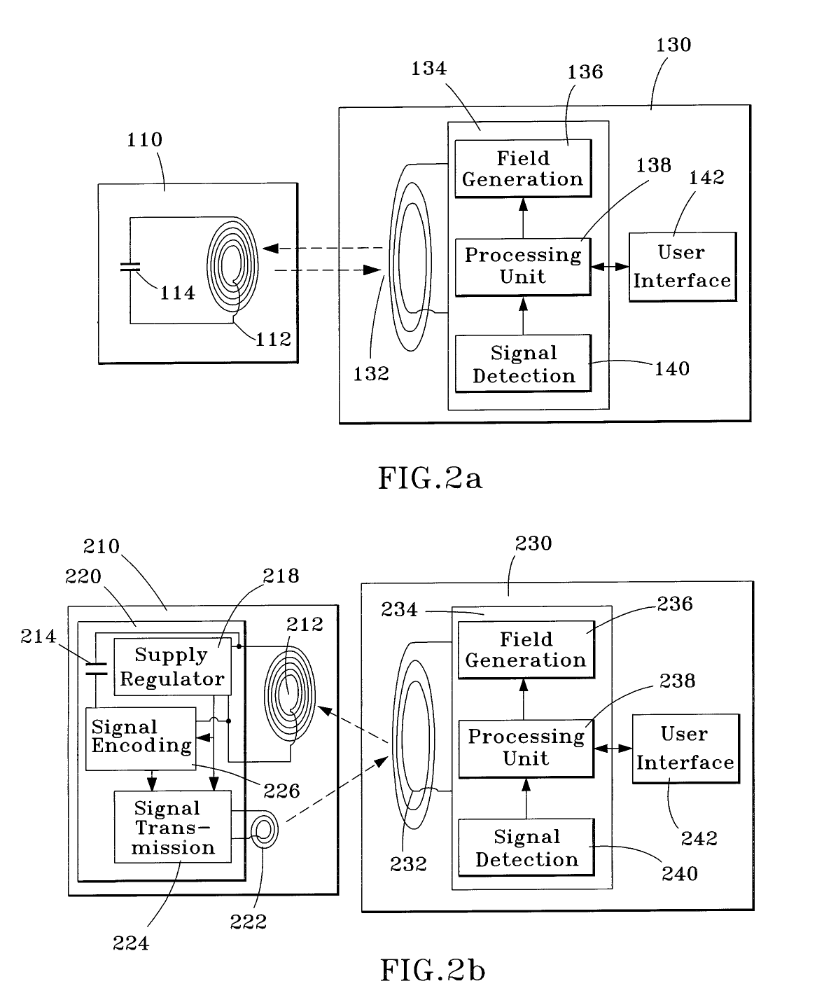

Illustrated in FIGS. 2a through 13 are various monitoring systems and components thereof that implement one or more implantable sensors whose outputs are derived from changes in inductance of an inductor. The inductor is in the form of a coil, such as a freestanding wire loop, a conductor on a flexible substrate, etc., and is implanted so as to be capable of monitoring a condition of a person or inanimate structure, including motion or strain of, for example, a soft tissue or organ such as a bladder, vessel, etc., or a rigid structure such as natural and artificial bones and joints, nonmedical structures formed of essentially any material, etc. The implantable sensors are preferably part of a wireless monitoring system that includes a non-implanted reader unit that allows for telemetric communications with the sensor. With such an approach, the invention provides telemetric monitoring systems for noninvasively monitoring parameters associated with conditions surrounding the implantable sensor, including conditions that reflect the health or a condition of a person or structure in which the sensor is implanted.

FIG. 2a represents a first embodiment of the invention as a monitoring system that includes a sensor implant 110 and a non-implanted reader unit 130 that are adapted for wireless communication. The implant 110 is preferably adapted for permanent (chronic) placement, such as within the human body, and wireless interrogation by the reader unit 130. If implanted within the human body, the implant 110 allows for the measurement and transmission, in real time, of various physiologic parameters. In the same manner, the integrity of the implant 110 can also be noninvasively monitored over time. In medical applications, the reader unit 130 enables a physician, caregiver, or patient to monitor the output of the implanted implant 110 at any time, including home care monitoring as well as in a hospital or physician's office. According to a preferred aspect of the invention, the implant 110 is shown in FIG. 2a without a battery, and therefore its operation does not require occasional replacement or charging of a battery. Instead, the energy required to perform the sensing operation is derived from the reader unit 130, as discussed in more detail below. However, the inclusion of a battery within the implant 110 is also within the scope of the invention

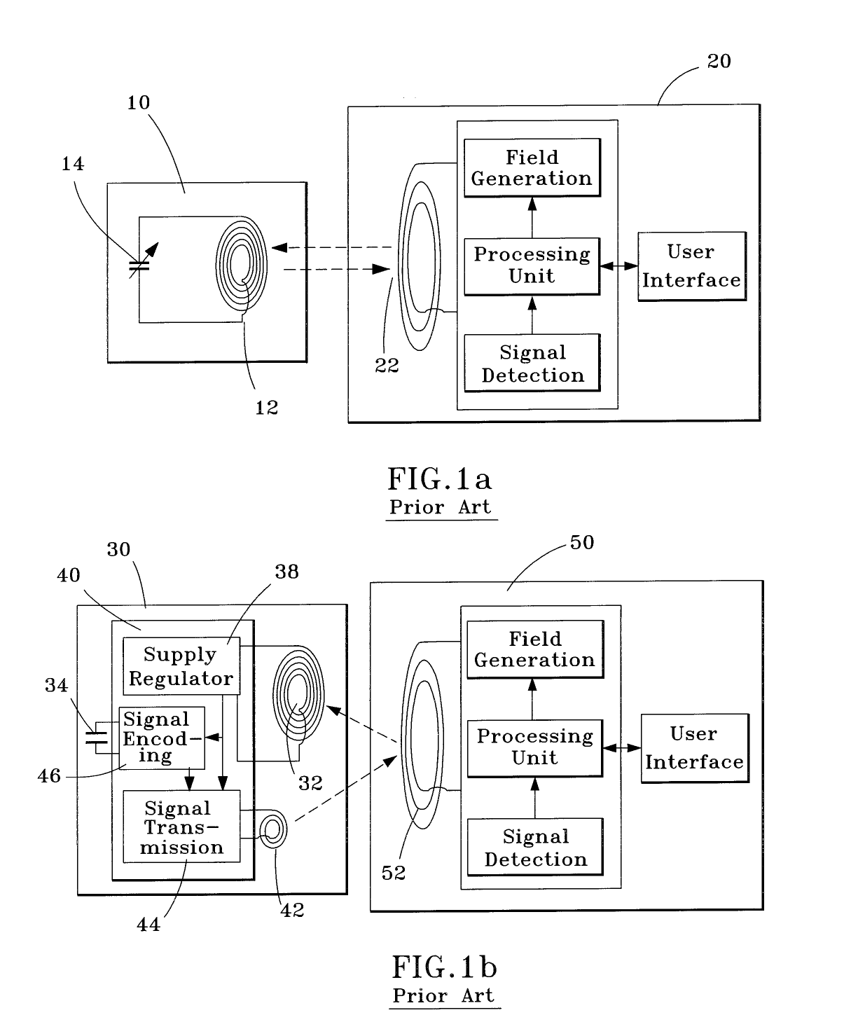

In FIG. 2a, the power for the implant 110 is wirelessly transmitted by the reader unit 130 through an electromagnetic or RF field. Such a batteryless, wireless telemetry link is implemented in FIG. 2a using a resonant scheme. For this approach, the implant 110 contains an inductor coil 112 and a fixed capacitor 114, which together form an LC circuit that has a specific resonant frequency. At that resonant frequency, the LC circuit presents a measurable change in magnetically coupled impedance load to a inductor coil 132 located within the reader unit 130. The resonant frequency of the LC circuit is a function of the inductance of the coil 112 and the capacitance of the sensor capacitor 114. Because the capacitor 114 is fixed and therefore has a fixed capacitance value, the reader unit 130 is adapted to monitor any changes in the coil 112 by sensing the resonant frequency of the circuit. As such, a fundamental difference between the implant 110 of the present invention and the prior art sensor 10 of FIG. 1a is that the implant 110 uses the coil 112 as a sensing element of the sensor implant 110, and as such the coil 112 can be referred to as a sensing coil 112. For this reason, and as described in more detail below, the sensing coil 112 is intentionally configured to be capable of physically responding to surrounding conditions, generally through deflection or other relative movement of a conductor within the coil 112, and in so doing the reader unit 130 is able to monitor strain, pressure, or other conditions capable of causing movement of the sensing coil 112 by monitoring the resonant frequency of the circuit.

In FIG. 2a, the reader unit 130 is represented as being adapted to communicate, power and monitor the sensor implant 110 using front-end electronics 134 that include field generation circuitry 136 for generating the electromagnetic or RF field transmitted by the coil 132, signal detection circuitry 140 for receiving the impedance signal reflected by the sensing coil 112, and a processing unit 138 that processes signals received through the detection circuitry 140, relays data to a user interface 142, and enables control of the field generation circuitry 136. The fabrication and operation of the front-end electronics 134 and its circuit components 136, 138, and 140 are well known in the art, and therefore will not be discussed in any detail here. The user interface 142 associated with the reader unit 130 may be a display, computer, or other data logging devices that can be physically coupled to the unit 130 or a separate remote unit.

A more preferred communication scheme based on magnetic or electromagnetic telemetry is represented in FIG. 2b, which depicts a sensor implant 210 similar to the prior art sensor 30 of FIG. 1b, but again with the notable exception that the sensing element of the implant 210 is not a mechanical capacitor, but an inductor. The implant 210 includes an integrated circuit (IC) chip 220 (such as an application specific integrated circuit, or ASIC) with on-board circuitry that preferably receives its operating power from an external source other than a battery. Such a batteryless implant 210 is referred to as a passive device, and in the absence of an external powering device lies passive without any internal means to power itself. When operation of the implant 210 is desired, a reader unit 230 with a power-transmitting capability is brought within suitable range of the implant 210.

In the embodiment shown in FIG. 2b, the reader unit 230 is represented as being adapted to power, monitor, and communicate with the sensor implant 210 using front-end electronics 234 that include field generation circuitry 236 for generating an alternating electromagnetic field, an inductor coil 232 for transmitting the alternating electromagnetic field to an inductor coil 212 of the implant 210, signal detection circuitry 240 for receiving data transmitted by a second inductor coil 222 of the implant 210, and a processing unit 236 that processes the data received through the detection circuitry 240, relays the processed data to a user interface 242, and enables control of the field generation circuitry 236. As with the readout unit 130 of FIG. 2a, the fabrication and operation of the front-end electronics 234 and its circuit components 236, 238, and 240 are well known in the art and therefore will not be discussed in any detail here, and the user interface 242 may be a display, computer, or other data logging devices that can be physically coupled to the unit 230 or a separate remote unit.

As those skilled in magnetic and electromagnetic telemetry are aware, a number of modulation schemes are available for transmitting data between the implant 210 and readout unit 230 via magnetic coupling. Preferred schemes include but are not limited to amplitude modulation, frequency modulation, frequency shift keying, phase shift keying, and also spread spectrum techniques. A preferred modulation scheme for a particular application may be determined by the specifications of the application, and is not intended to be limited under this invention. In addition, there are many technologies developed that allow the implant 210 to communicate the signals back to the reader unit 230. It should be further understood that the reader unit 230 may transmit either a continuous level of RF power to supply the energy for the implant 210, or it may pulse the power allowing temporary storage in a battery or capacitor (e.g., 214) on the implant 210. Similarly, the implant 210 may signal back to the reader unit 230 at any interval in time, delayed or instantaneous, during reader unit RF transmission or alternately in the absence of reader transmission.

When sufficient alternating voltage has been induced by the reader unit 230 on the inductor coil 212 of the implant 210, a rectification circuit 218 on the IC chip 220 converts the alternating voltage into a direct voltage that can be used by the IC chip 220 as a power supply for signal conversion and communication. The electronic circuitry on the IC chip 220 is represented as further including a signal conditioning circuit 226 and a signal transmission circuit 224, both of which are powered by the rectification circuit 218. Finally, the implant 210 includes a fixed capacitor 214 having a fixed capacitive output, which is electrically coupled to the inductor coil 212 to form an LC circuit. As with the embodiment of FIG. 2a, at its resonant frequency the LC circuit presents a measurable change in magnetically coupled impedance load to the coil 232 of the reader unit 230.

As previously noted, the implant 210 uses an inductor as a sensing element for the monitoring system. In the implementation of the invention shown in FIG. 2b, the implant 210 uses the inductor coil 212 to not only receive power from the reader unit 230, but also as the sensing element, such that the coil 212 will be referred to as a sensing coil 212. As such, and as discussed in more detail below, the sensing coil 212 is intentionally configured to be able to physically respond to surrounding conditions such as strain, pressure, etc., generally through deflection or other relative movement of portions of its conductor. The sensing coil 212 is connected to the signal conditioning circuit 226 on the IC chip 220, such that changes in the inductance of the sensing coil 212 are detected and processed by the signal conditioning circuit 226, which in turn prepares the processed output signal for transmission to the readout unit 230 via the signal transmission circuit 224 and coil 222. In this manner, the reader unit 230 is able to monitor strain, pressure, or other conditions capable of causing movement of the sensing coil 212 by monitoring the output of the implant 210.

Efficient implementations of the rectification, signal transmission, and signal conditioning circuits 218, 224, and 226 include standard electronic techniques. The rectification circuit 218 may be a full-bridge or half-bridge diode rectifier, and may include a capacitor for transient energy storage to reduce the noise ripple on the output supply voltage. As represented in FIG. 2b, the rectification circuit 218 may be implemented on the same chip 220 as the other circuits 224 and 226. The signal conditioning circuit 226 processes the output signal of the implant 210 by digitizing the inductance signal from the sensing coil 212 for RF transmission. Many different signal conditioning circuits are known in the art for this purpose. The signal transmission circuit 224 transmits the encoded signal from the signal conditioning circuit 226 for reception by the reader unit 230 by generating an alternating electromagnetic field that propagates from the inductor coil 222 to the reader unit 230.

When sufficient alternating voltage has been induced by the reader unit 230 on the inductor coil 212 of the implant 210 to enable the rectification circuit 218 to generate a sufficient level of direct voltage for signal conversion and communication, the implant 210 is considered alert and, in the preferred embodiment, also ready for commands from the reader unit 230. The maximum achievable distance is primarily limited by the electromagnetic field strength necessary to turn the implant 210 on. Another option, particularly useful for (but not limited to) situations in which long-term data acquisition is desired without continuous use of the readout unit 230, is to implement the implant 210 using an active scheme, such as by incorporating an additional capacitor, battery (primary or rechargeable), or other power-storage element that allows the implant 210 to function without requiring the immediate presence of the readout unit 230 as a power supply. With such an approach, data may be stored in the implant 210 and downloaded intermittently using the readout unit 230 as required.

The sensor implants 110 and 210 of this invention can be physically realized with a combination of any of several technologies, including those using microfabrication technology such as microelectromechanical systems (MEMS). The implants 110 and 210 may be fabricated so that, aside from the sensing coils 112 and 212, their components are enclosed in a hermetic sensor package formed by, for example, anodically bonded layers of glass and silicon (doped or undoped), which advantageously are biocompatible and therefore enable the implants 110 and 210 to be permanently (chronically) placed in a patient without any additional packaging. Anchoring of the implants 110 and 210 can be achieved with the sensing coils 112 and 212, though anchoring provisions may also be incorporated directly into the implant package or added through an additional assembly step in which an anchor is attached to the package.

A large number of possible geometries and structures are available for the sensing coils 112 and 212, which must be sufficiently flexible to physically react to external conditions, including strain, pressure, or other conditions capable of causing movement of the conductor that forms the coil 112/212. The conductors are preferably formed at least in part of high-conductivity biocompatible material, such as platinum, titanium, silver, gold, or another metal, alloy or conductive material. The conductors may also be protected with a biocompatible coating, such as a biocompatible dielectric material including parylene, PTFE, polyethylene, or silicone. The conductors may be in the form of a freestanding wire or filament, or conductive lines on a flexible substrate that can be attached to a structure to be monitored, or embedded in a structure or in a surface layer of a structure to be monitored. Such conductors can be formed by deposition techniques including sputtering, electroplating, lift-off, screen printing, or another technique known in the art.

As discussed below, one or more conductors of the coils 112 and 212 may be wound around a ferrite core to enhance magnetic properties, or formed into a long and thin or short and wide cylindrical solenoid or bladder shaped cover. To ensure that the conductors consistently respond to physical changes in the structure being monitored, the coils 112 and 212 are preferably attached in at least two places on a structure to be monitored, such as with adhesives, screws, tabs, ties, wires, sutures, or other attachment methods. The coils 112 and 212 may be totally or partially wrap around certain structures such as a vessel, aneurism, bone, bladder, or other internal organ (both artificial and natural) of a person, or embedded in certain structures such as artificial joints, bones, and organs (both artificial and natural). In each case, the coil 112/212 can be physically coupled to the structure it monitors so that at least portions of its shape are altered in response to changes in the shape or strain of the structure. For example, as the structure bends, swells or breaks, one or more gaps between portions of the coil conductor will change, altering the inductance of the coil 112/212 and hence resonance frequency of the sensor implant 110/210.

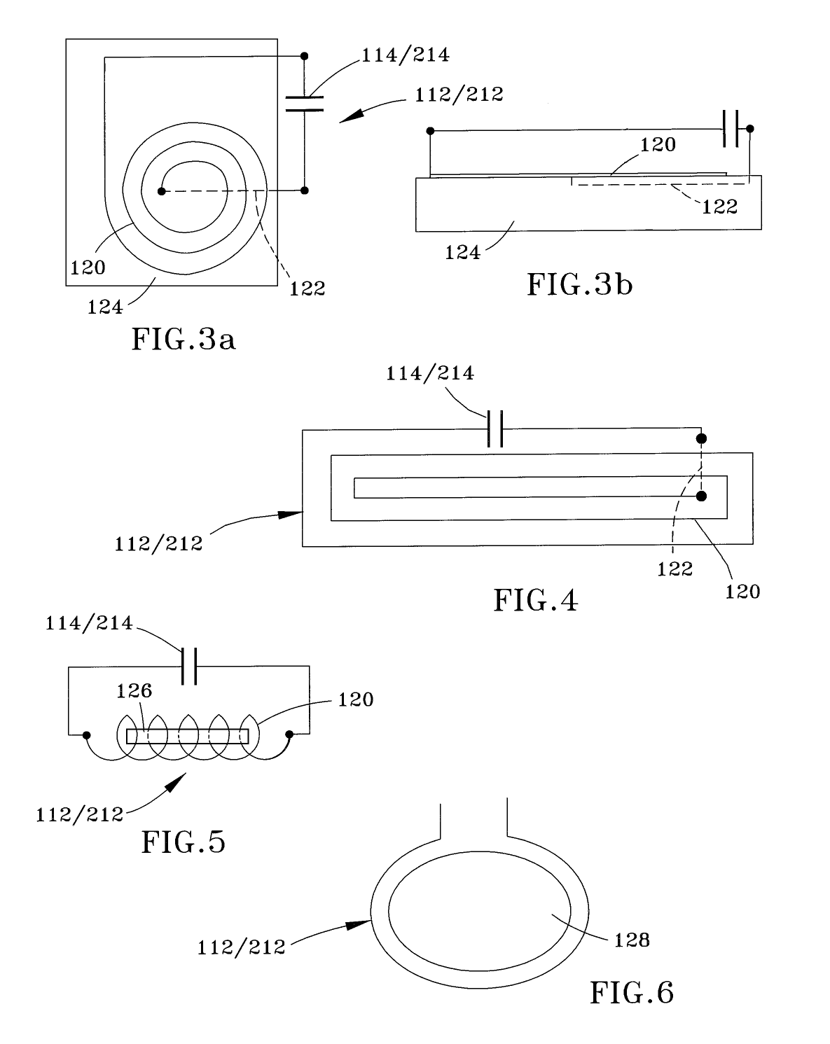

FIGS. 3a through 6 depict some of the coil shapes discussed above (the signal conditioning circuit 226 and other devices of the IC chip 220 are omitted in FIGS. 3a through 6 as a matter of convenience). FIGS. 3a, 3b, and 4 depict what may generally be termed a two-dimensional coil 112/212, in that the conductors 120 are primarily located in a single plane, the exception being a crossunder 122 embedded beneath the surface of a flexible substrate 124 in/on which the coil 112/212 is formed. Two-dimensional coils 112/212 of the type shown in FIGS. 3a, 3b, and 4 can be deposited on a flat substrate 124 yet acquire a three-dimensional shape by flexing the substrate 124, such as when the substrate 124 is attached to a nonplanar surface. FIG. 5 depicts what may be termed a three-dimensional coil 112/212, in that the as-produced conductor 120 does not lie in a single plane as a result of, for example, being deposited on or wrapped around a three-dimensional object. FIG. 5 illustrates that a ferrite core 126 can be inserted within an opening formed by the conductor 120 so as to be surrounded by the coil 112/212 to increase its inductance. The coil 112/212 must be free to move relative to the ferrite core 126, and as such the core 126 is preferably embedded separately and independently from the coil 112/212 in a structure to be monitored by the implant 110/210.

As represented in FIG. 6, the coils 112/212 of FIGS. 3a through 5 can be used to monitor the expansion or contraction of a pliable structure 128, such as a vessel (blood vessel, etc.), aneurysm, bladder (artificial or natural), or other organ around which the coil 112/212 or the substrate 124 carrying the coil 112/212 can be wrapped. In a similar manner, the coil 112/212 can be used to sense pressure or force via an inductance change resulting from changes in the gap or gaps between adjacent strands of the conductor 120 or between the conductor 120 and the ferrite core 126. In this manner, the implant 110/210 operates as a wireless pressure or force sensor that is based on an inductance change, instead of a capacitance change sensed by the implants 10 and 30 of the prior art.

In the coils 112/212 depicted in FIGS. 3a through 5, the capacitor 114/214 is represented as being an integral component of the conductor 120, though such is not a requirement. Because the capacitor 114/214 is not required to respond to an external condition, the capacitor 114/214 can be a fixed component soldered to or overlying the conductor 120 of the substrate 124 in/on which the coil 112/212 is formed. The capacitor 114/214 can be provided with a stiffening element (not shown) to inhibit the capacitor 114/214 from flexing with the substrate 124 and its coil 112/212.

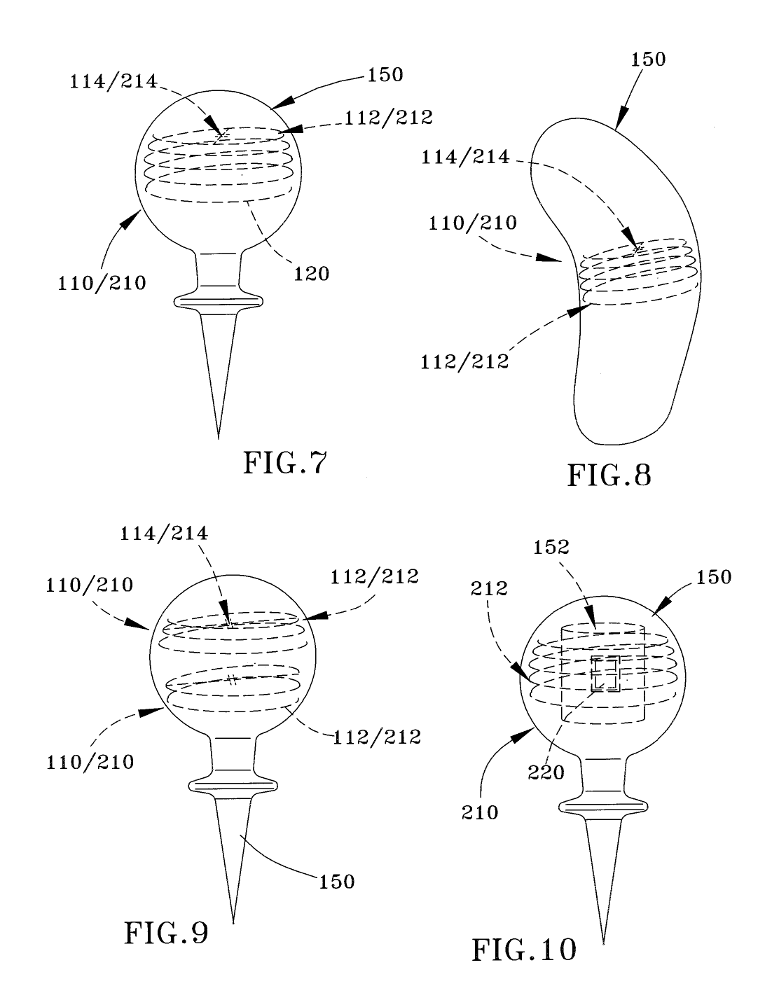

FIGS. 7 through 10 depict implants 110/210 of this invention incorporated into rigid structures 150, such as an artificial ball joint for the hip or shoulder (FIGS. 7, 9, and 10) and an artificial patella for a knee (FIG. 8), for the purpose of monitoring stress, wear, inflammation, infection, etc., of the rigid structures 150. The implants 110/210 are placed such that mechanical stresses and forces on the rigid structures 150 will affect the spacing between portions of the conductors 120 of the coils 112/212, leading to an inductance change that can be detected with the readout unit 130/230. The structure 150 may be formed of a dielectric material in which the coil 112/212 of the implant 110/210 is embedded, or have a dielectric surface coating in or on which the coil 112/212 is present and preferably embedded. Suitable dielectric materials include PTFE, ceramics, glass, calcium carbonate, artificial bone, polyethylene or other plastic or polymers, and combinations of these types of materials. The coil 112/212 can also be applied to or embedded in an implanted reinforcement structure, such as a rod or plate, that is attached to the rigid structure 150 (notable examples include bones of the leg, arm, hip, skull, and spine) to monitor the condition of the reinforcement structure. The capacitor 114/214 and/or the IC chip 220 can also be embedded in the structure 150, preferably encased within a soft compliant material to reduce mechanical stresses that would be imposed by the structure 150 on these elements. In each of FIGS. 7 through 10, it is evident that the coil 112/212 is likely to have a much larger diameter than that possible for the fixed on-chip inductor coils 12, 32, and 42 used in the prior art of FIGS. 1a and 1b, and as such are capable of a longer transmission range than such prior art coils.

In FIG. 8, the implant 110/210 can be used as a knee joint pressure sensor by sensing pressure in the synovial fluid surrounding the knee joint. In FIG. 9, multiple implants 110/210 are shown as being implanted to monitor different areas of the same structure 150. Adjacent implants 110/210 preferably operate at different frequencies to enable them to be independently monitored with a readout unit 130 and 230. In FIG. 10, a metal core 152 is embedded in the rigid structure 150 to act as a magnetic core that increases the range of the implant 210. FIG. 10 further shows the IC chip 220 as being embedded in the structure 150 and encased within a soft compliant material.

With the embodiments shown in FIGS. 3a through 10, the coils 112/212 of the implants 110/210 can also be used to measure the conductivity, dielectric constant, temperature, and/or pH of the region surrounding the implant 110/210. Such a capability has applications for monitoring swelling, inflammation, infection, particle detection, and changes in interstitial fluid pressure. These functional aspects of the invention may be achieved by exposing one or more portions of the coil 112/212 to the surrounding tissue or fluid, such that a conductive, capacitive or dielectric path is created between adjacent portions of the coil 112/212. The implants 110 and 210 can also include or otherwise be combined with other sensing elements, examples of which include pressure, gas (e.g., oxygen, carbon dioxide, etc.), temperature, chemical (e.g., glucose), pH, flow, and velocity sensors. Various miniaturized examples of such sensors are known to those skilled in the art, and any one or more of these sensors can be utilized in or with the sensor implants 110/210 of the present invention.

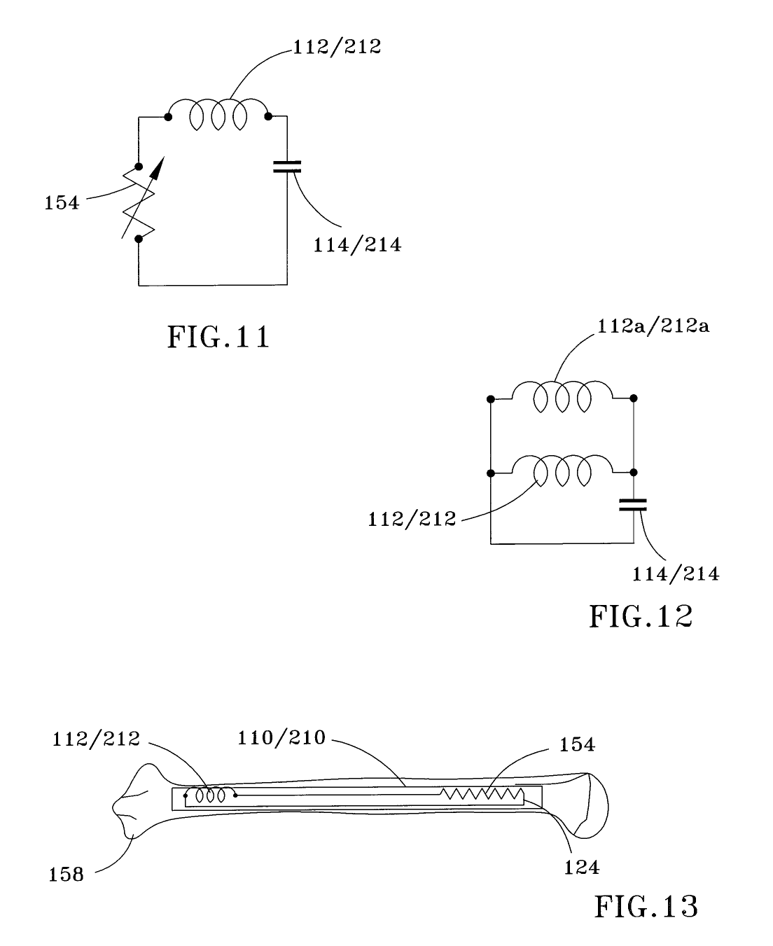

FIGS. 11 through 13 represent other manifestations of the invention, including the inclusion of a resistor 154 (FIG. 11) and an additional inductor coil 112A/212A (FIG. 12) in the circuit containing the inductor coil 112/212 (the signal conditioning circuit 226 and other devices of the IC chip 220 are omitted in FIGS. 11 and 12 as a matter of convenience). In FIG. 11, the resistor 154 is in series with the sensing coil 112/212 and capacitor 114/214, forming an LCR circuit. FIG. 11 also indicates the resistor 154 as having a variable resistance. In FIG. 13, an implant 110/210 with the LCR circuit of FIG. 11 is shown supported on a flexible substrate 124 attached to a long bone 158 (for example, the femur). The variable resistor 154 is in the form of a strain gage arranged to be responsive to stresses and strains within the bone 158, such that the output of the implant 110/210 is based on the effects that strains, stresses, bending, etc., have on the inductance and resistance of the sensing coil 112/212 and resistor 154, respectively.

In FIG. 12, the inductor coil 112A/212A can be located close to a surface of the body in which the implant 110/210 is implanted to improve the transmission range of the implant 110/210. For example, the inductor coil 112A/212A, which is in addition to the coil 112 of FIG. 2a and in addition to the coils 212 and 222 of FIG. 2b, can be connected in parallel (as shown in FIG. 12) or in series with the sensing coil 112/212 and internally located in closer proximity to the skin of the patient than the sensing coil 112/212, or externally located and coupled via a skin piercing cable to the implant 110/210. Placing the coil 112A/212A more accessible to the readout unit 130/230 reduces attenuation of the output signal of the implant 110/210 by the body of a patient or reduces shielding of the RF signal by an object containing, coated with, or formed of an electrically conductive material. The ability to place the coil 112A/212A near or at the exterior of the body being monitored also adds to the flexibility and use of the implant 110/210. For example, the implant 110/210 can be used in brain, cardiac, and orthopedic applications where the sensing coil 112/212 is located within the interior of the patient, such as in an organ, limb, joint, or prosthetic device, and the coil 112A/212A is subdermal or located close to the skin surface. For example, the coil 112A/212A can be subdermal and sutured to reduce infection.

While the specific type of implant 110/210 chosen for a given application will depend on the particular application, in all cases the implant 110/210 can be of a sufficiently small size to facilitate placement within a catheter for delivery and implantation, or surgically implanted, or built into artificial bone, joints and organs prior to surgical implantation of these devices.

The implant 110/210 and/or its readout unit 130/230 can also include the operation of algorithms that account for various factors that might alter the output of the implant 110/210, such as pressure or strain changes due to the position, weight, and body temperature of the patient. Parameters such as the patient name, current weight, weight at the time of surgery, body temperature, blood pressure, and posture can all be entered into the reader unit 130/230 before a measurement is taken to assist in obtaining an accurate reading and appropriate decision about the integrity of the implant 110/210.

In addition to the implants 110 and 210 and reader units 130 and 230 described above, monitoring systems of this invention can be combined with other technologies to achieve additional functionalities. For example, the monitoring system can be implemented to have a remote capability, such as home monitoring that may employ telephone, wireless communication, or web based delivery of information received from the implant 110/210 by the reader unit 130/230 to a physician or caregiver.

While the invention has been described in terms of specific embodiments, it is apparent that other forms could be adopted by one skilled in the art. Therefore, the scope of the invention is to be limited only by the following claims.

1. A monitoring system comprising:
at least one sensing device adapted to be implanted in a body and attached to a structure within the body, the sensing device comprising an electrical circuit containing at least a first inductor coil that comprises a conductor with portions thereof separated by gaps, the first inductor coil being adapted to be physically coupled to the structure so that changes in shape and size of the structure cause changes in shape and/or size of the first inductor coil and/or changes in the gaps so as to alter the inductance of the first inductor coil when current flows through the electrical circuit; and
a readout device that is not adapted to be implanted in the patient, the readout device comprising at least one inductor coil and telemetric means for at least one of electromagnetic telecommunication and electromagnetic powering of the sensing device with the inductor coil thereof.

2. The monitoring system according to claim 1, wherein the sensing device further comprises a fixed capacitor.

3. The monitoring system according to claim 2, wherein the fixed capacitor is in series with the first inductor coil in the electrical circuit, and the electrical circuit has a resonant frequency that is dependent on the inductance of the first inductor coil.

4. The monitoring system according to claim 3, wherein the electrical circuit of the sensing device further comprises a variable resistor n series with the fixed capacitor and the first inductor coil so as to form an LCR circuit therewith.

5. The monitoring system according to claim 4, wherein the variable resistor is a strain gage arranged within the sensing device to be responsive to stresses and strains within the structure.

6. The monitoring system according to claim 3, wherein the inductor coil of the readout device electromagnetically telecommunicates with and electromagnetically powers the sensing device through the first inductor coil of the sensing device.

7. The monitoring system according to claim 1, wherein the electrical circuit of the sensing device further comprises a strain gage in series with the first inductor coil and arranged within the sensing device to be responsive to stresses and strains within the structure.

8. The monitoring system according to claim 1, wherein the electrical circuit of the sensing device further comprises a second inductor coil electrically coupled to the first inductor coil for being electromagnetically powered by the readout device.

9. The monitoring system according to claim 1, wherein the sensing device further comprises signal processing circuitry electrically coupled to the first inductor coil and adapted to convert the inductance of the first inductor coil to an output signal that can be transmitted to the inductor coil of the readout device.

10. The monitoring system according to claim 9, wherein the sensing device is electromagnetically powered by the inductor coil of the readout device through the first inductor coil of the sensing device, and the sensing device further comprises a second inductor coil electrically coupled to the signal processing circuitry and adapted to transmit the output signal to the inductor coil of the readout device.

11. The monitoring system according to claim 1, wherein the first inductor coil has a two-dimensional geometry.

12. The monitoring system according to claim 11, wherein the sensing device comprises a flexible substrate that supports the first inductor coil, the flexible substrate being adapted to be attached to the structure.

13. The monitoring system according to claim 1, wherein the first inductor coil has a three-dimensional geometry.

14. The monitoring system according to claim 13, wherein the first inductor coil is a freestanding coil adapted to be wrapped around the structure.

15. The monitoring system according to claim 13, wherein the first inductor coil is embedded within the structure.

16. The monitoring system according to claim 13, wherein the first inductor coil is embedded within a coating on the surface of the structure.

17. The monitoring system according to claim 13, further comprising a metal or ferrite core within the structure and surrounded by the first inductor coil.

18. The monitoring system according to claim 1, wherein the structure is an artificial bone or joint.

19. The monitoring system according to claim 1, wherein the structure is a pliable organ.

20. The monitoring system according to claim 19, wherein the structure is a bladder.

21. The monitoring system according to claim 1, wherein the sensing device is attached to an implantable reinforcement structure adapted to be attached to the structure, such that the sensing device is coupled to the structure through the implantable reinforcement structure.
